(19) United States
(12) Patent Application Publication
(10) Pub. No.: US 2010/0047197 A1
(43) Pub. Date: Feb. 25, 2010

(54) COSMETIC FORMULATION CONTANING GLUCOSYL GLYCERIDES AND UREA

(75) Inventors: Cathrin Scherner, Norderstedt (DE); Rainer Kroepke, Schenefeld (DE); Ute Breitenbach, Hamburg (DE); Alexander Filbry, Hamburg (DE); Cornelia Gatermann, Hamburg (DE)

Correspondence Address: GREENBLUM & BERNSTEIN, P.L.C., 1950 ROLAND CLARKE PLACE, RESTON, VA, 20191, US

(73) Assignee: BEIERSDORF AG, Hamburg (DE)

(21) Appl. No.: 12/515,150

(22) PCT Filed: Nov. 9, 2007
(86) PCT No.: PCT/EP2007/009730

§ 371 Date: Aug. 31, 2009

(30) Foreign Application Priority Data
Nov. 17, 2006 (DE) 10 2006 055 044.7

(51) Int. Cl. A61K 8/60; A61Q 19/00; A61K 8/06; A61Q 17/04; A61K 31/122

(52) U.S. Cl. 424/59; 514/25; 424/60; 424/94.1

(57) ABSTRACT

Cosmetic preparation containing a combination of a) urea and b) one or more glucosyl glycerides.

The present invention relates to a cosmetic preparation with urea and one or more glucosyl glycerides.

The desire to look beautiful and attractive is deeply engrained in human nature. Even though the ideal of beauty has changed over time, people have always tried to attain a faultless appearance. The condition and the appearance of the skin is a significant part of a beautiful and attractive outward appearance.

In order for the skin to be able to fulfill its biological function fully, it needs regular cleansing and care. The cleansing of the skin serves to remove dirt, sweat and the residue of dead skin particles that provide an ideal breeding ground for germs and parasites of all kinds. Skin care products are used mostly for moisturizing and regreasing the skin. Active substances are often added thereto that regenerate the skin and, e.g., are designed to prevent and reduce its premature aging (e.g., the formation of fine lines, wrinkles).

The active ingredients used in cosmetics include, inter alia, glucosyl glycerides. They moisturize the skin and intensify its barrier function. However, the preparations known according to the prior art that contain glucosyl glycerides have the disadvantage of slowly dislocating in the weakly acid pH range which is of physiological interest, so that the storage life of the preparations leaves something to be desired.

It was therefore the object of the present invention to eliminate the disadvantages of the prior art and to develop cosmetic preparations containing glucosyl glycerides that are storage stable.

The object is surprisingly attained through a cosmetic preparation containing a combination of

a) urea and

b) one or more glucosyl glycerides.

Embodiments of the present invention that are advantageous according to the invention are characterized in that the preparation according to the invention contains the glucosyl glyceride(s) in a concentration of from 0.01 to 10% by weight, preferably in a concentration of from 0.05 to 6% by weight and particularly preferably in a concentration of from 0.1 to 3% by weight, in each case based on the total weight of the preparation.

It is advantageous for the purposes of the present invention if the preparation according to the invention contains urea in a concentration of from 0.001 to 10% by weight, preferably in a concentration of from 0.1 to 6% by weight, and particularly preferably in a concentration of from 1 to 4% by weight, in each case based on the total weight of the preparation.

It is advantageous according to the invention if the preparation is free of (2-hydroxyethyl)urea.

Glucosyl glycerides having the formula

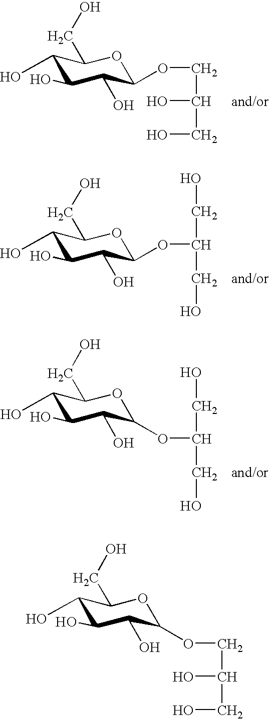

are advantageous for the purposes of the present invention.

Although DE 195 40 749 and the unpublished DE 10 2005 023634, DE 10 2005 023635, DE 10 2005 023636, DE 10 2005 023637, DE 10 2005 023638, DE 10 2005 023639, DE 10 2005 023640, DE 10 2005 023641, DE 10 2006 034530.4 and DE 20 2006 011472.6 are described in the prior art, as well as JP 2004-331581, JP 2004-331576, JP 2004-331578, JP 2004-331579, JP 2004-331580, JP 2004-331582, JP 2004-331583, which are hereby incorporated as reference, these documents could not show the way to the present invention.

The preparation according to the invention is advantageously present according to the invention in the form of a gel, an emulsion or a dispersion. It is thereby preferred according to the invention for the preparation according to the invention to be present in the form of an emulsion.

In an embodiment of the present invention that is particularly preferred according to the invention, the preparation is present in the form of an O/W emulsion.

In this case, it is preferred according to the invention when the preparation contains one or more O/W emulsifiers selected from the group of the compounds glyceryl stearate citrate, glyceryl stearate, cetearyl glucoside, stearic acid and salts thereof, polyglyceryl-3-methylglycose distearate, cetearath-20, PEG-40 stearate, PEG-100 stearate, sodium cetearyl sulfate.

According to the invention, these O/W emulsifiers can advantageously be contained in the preparation in a concentration of from 0.001 to 10% by weight and preferably in a concentration of from 0.1 to 7% by weight, based on the total weight of the preparation.

Another embodiment of the present invention that is preferred according to the invention is characterized in that the preparation is present in the form of a W/O emulsion.

With this embodiment it is preferred according to the invention when the preparation contains one or more W/O emulsifiers selected from the group of the compounds polyglyceryl-2-dipolyhydroxystearate, PEG-30 dipolyhydroxystearate, cetyl dimethicone copolyol, polyglyceryl-3 diisostearate.

These W/O emulsifiers according to the invention can be advantageously contained according to the invention in a concentration of from 0.1 to 10% by weight and preferably in a concentration of from 0.2 to 7% by weight, based on the total weight of the preparation.

Embodiments of the present invention that are advantageous according to the invention are characterized in that the preparation contains one or more further UV filters selected from the group of the compounds phenylene-1,4-bis-2-benzimidazyl)-3,3′-5,5′-tetrasulfonic acid salts; 2-phenylbenzimidazole-5-sulfonic acid salts; 1,4-di(2-oxo-10-sulfo-3-bornylidenemethyl)-benzene and salts thereof, 4-(2-oxo-3-bornylidenemethyl)benzenesulfonic acid salts; 2-methyl-5-(2-oxo-3-bornylidenemethyl)sulfonic acid salts; 2,2′-methylene-bis-(6-(2H-benzotriazole-2-yl)-4-(1,1,3,3-tetramethylbutyl)-phenol); 2-(2H-benzotriazole-2-yl)-4-methyl-6-[2-methyl-3-[1,3,3,3-tetramethyl-1-[(trimethylsilyl)oxy]disiloxanyl]propyl]-phenol; 3-(4-methylbenzylidene)campher; 3-benzylidene campher; ethylhexyl salicylate; terephthalidene dicamphorsulfonic acid; (2-ethylhexyl)4-(dimethylamino)benzoate; amyl 4-(dimethylamino)benzoate; di(2-ethylhexyl)4-methoxybenzalmalonate; (2-ethylhexyl)4-methoxycinnamate; isoamyl 4-methoxycinnamate; 2-hydroxy-4-methoxybenzophenone, 2-hydroxy-4-methoxy-4′-methylbenzophenone; 2,2′-dihydroxy-4-methoxybenzophenone; hexyl 2-(4′-diethylamino-2′-hydroxybenzoyl)benzoate, 4-(tert.-butyl)-4′-methoxydibenzoylmethane; homomethyl salicylate; 2-ethylhexyl 2-hydroxybenzoate; 2-ethylhexyl-2-cyano-3,3-diphenylacrylate; dimethicodiethylbenzalmalonate; 3-(4-(2,2-bis-ethoxycarbonylvinyl)-phenoxy)propenyl)-methoxysiloxane/dimethylsiloxane copolymer; 2,4-bis-{[4-(2-ethylhexyloxy)-2-hydroxy]-phenyl}-6-(4-methoxyphenyl)-1,3,5-triazine (INCI: bis-ethylhexyloxyphenol methoxyphenyl triazine); dioctylbutylamidotriazone (INCI: diethylhexyl-butamidotriazone); 2,4-bis-[5-1 (dimethylpropyl)benzoxazole-2-yl-(4-phenyl)-imino]-6-(2-ethylhexyl)-imino-1,3,5-triazine with the (CAS Nr. 288254-16-0); tris(2-ethylhexyl) 4,4′,4″-(1,3,5-triazine-2,4,6-triyltriimino)-tris-benzoate (also: 2,4,6-tris-[anilino-(p-carbo-2′-ethyl-1′-hexyloxy)]-1,3,5-triazine (INCI: ethylhexyl triazone); 2,4,6-tribiphenyl-4-yl-1,3,5-triazine; merocyanine; titanium dioxide, zinc oxide in a concentration of from 0.01 to 40% by weight and preferably in a concentration of from 1 to 20% by weight, based on the total weight of the preparation.

It is advantageous according to the present invention if the preparation according to the invention is free from p-methylbenzylidene camphor.

The pigments (titanium dioxide, zinc oxide) can be used advantageously for the purposes of the present invention also in the form of commercially available oily or aqueous predispersions. Dispersants and/or solubilizers can advantageously be added to these predispersions.

Advantageously according to the invention the pigments (titanium dioxide, zinc oxide) can be surface treated (coated), thus forming or maintaining, for example, a hydrophilic, amphiphilic or hydrophobic character. This surface treatment can comprise the pigments being provided with a thin hydrophilic and/or hydrophobic inorganic and/or organic layer according to processes known per se. The different surface coatings can also contain water in the sense of the present invention.

Inorganic surface coatings in the sense of the present invention can comprise aluminum oxide (Al₂O₃), aluminum hydroxide Al(OH)₃, or aluminum oxide hydrate (also: alumina, CAS No.: 1333-84-2), sodium hexametaphosphate (NaPO₃)₆, sodium metaphosphate (NaPO₃)ₙ, silicon dioxide (SiO₂) (also: silica, CAS No.: 7631-86-9), barium sulfate (BaSO₄) or iron oxide (Fe₂O₃). These inorganic surface coatings can occur in combination and/or in combination with organic coating materials.

Organic surface coatings for the purposes of the present invention can comprise plant or animal aluminum stearate, plant or animal stearic acid, lauric acid, dimethylpolysiloxane (also dimethicone), methylpolysiloxane (methicone), simethicone (a mixture of dimethylpolysiloxane with an average chain length of 200 to 350 dimethylsiloxane units and silica gel) or alginic acid. These organic surface coatings can occur alone, in combination and/or in combination with inorganic coating materials.

Embodiments of the present invention that are advantageous according to the invention are characterized in that the preparation according to the invention contains one or more compounds selected from the group of parabens, phenoxyethanol, ethylhexylglycerin, 2-methylpropane-1,3-diol, butylene glycol, propylene glycol, which can be present in the preparation in a total concentration of from 0.01 to 10% by weight based on the total weight of the preparation.

Advantageously according to the invention the preparation according to the invention contains as further ingredients one or more compounds selected from the group of the compounds folic acid, phytoene, D-biotin, coenzyme Q10, alpha-glucosylrutin, carnitine, polydocanol, natural and/or synthetic isoflavonoids, in particular genistein, flavonoids, carotenoids, creatine, creatinine, taurine, ascorbic acid+derivatives oxygen, tocopherol+esters, dihydroxyacetone; 8-hexadecene-1,16-dicarboxylic acid, long-chain and short-chain hyaluronic acid (i.e., hyaluronic acid with an average molecular weight of 1 million to 3 million Dalton, as well as 5000 Dalton—1 million Dalton) and/or licochalcone A. Ingredients of this type can be contained in the preparation in an individual concentration of from 0.01 to 10% by weight, based on the total weight of the preparation. The use of carnitine is thereby particularly preferred according to the invention.

In addition, the preparations according to the present invention can advantageously also comprise repellents for protection against mosquitos, ticks and spiders and the like. For example, N,N-diethyl-3-methylbenzamide (trade name: “DEET”), dimethyl phthalate (trade name: Palatinol M, DMP), 1-piperidinecarboxylic acid-2-(2-hydroxyethyl)-1-methylpropyl ester and in particular ethyl 3-(N-n-butyl-N-acetylamino)propionate (available under the trade name Insekt Repellent® 3535 from Merck) are advantageous. The repellents can either be used individually or in combination.

Moisturizers is the term used to refer to substances or mixtures of substances which impart to cosmetic preparations the property, following application or distribution on the surface of the skin, of reducing moisture release from the horny layer (also called trans-epidermal water loss (TEWL)) and/or of positively influencing hydration of the horny layer.

Advantageous moisturizers within the meaning of the present invention are, for example, glycerin, lactic acid and/or lactates, in particular sodium lactate, butylene glycol, propylene glycol, panthenol, biosaccharide gum-1, glycine soya, ethylhexyloxyglycerin, pyrrolidonecarboxylic acid and hyaluronic acid. In addition, it is particularly advantageous to use polymeric moisturizers from the group of water-soluble and/or water-swellable and/or water-gellable polysaccharides. A fucose-rich polysaccharide, which is filed in the Chemical Abstracts under the Registry number 178463-23-5 and which is available, for example, under the name Fucogel® 1000 from SOLABIA S. A., for example, is particularly advantageous. Moisturizers can advantageously also be used as anti-wrinkle active ingredients for the protection from changes in the skin, such as arise, for example, during skin aging.

It is advantageous for the purposes of the present invention for the preparation according to the invention to contain one or more humectants in a total concentration of from 0.1 to 20% by weight and preferably in a total concentration of from 0.5 to 10% by weight, in each case based on the total weight of the preparation.

The cosmetic preparations according to the invention can also advantageously, but not necessarily, comprise fillers which, for example, further improve the sensory and cosmetic properties of the formulations and, for example, bring about or enhance a velvety or silky skin feel. Advantageous fillers within the meaning of the present invention are starch and starch derivatives (such as, for example, tapioca starch, distarch phosphate, aluminum or sodium starch octenylsuccinate and the like), pigments which have neither primarily a UV filter effect nor a coloring effect (such as, for example, boron nitride etc.) and/or Aerosils® (CAS No. 7631-86-9) and/or talc.

The water phase of the preparations according to the invention can advantageously comprise customary cosmetic auxiliaries, such as, for example, alcohols, in particular those of low C number, preferably ethanol and isopropanol, diols or polyols of low C number and ethers thereof, preferably propylene glycol, 2-methylpropane-1,3-diol, ethylene glycol, ethylene glycolmonoethyl or monobutylether, propylene glycol monomethyl, monoethyl or monobutylether, diethylene glycol monomethyl or monoethylether and analogous products, polymers, foam stabilizers, electrolytes, and in particular one or more thickeners, which may advantageously be chosen from the group silicon dioxide, aluminum silicates, polysaccharides or derivatives thereof, e.g. hyaluronic acid, xanthan gum, carrageenans, hydroxypropylmethylcellulose, particularly advantageously from the group of polyacrylates, preferably a polyacrylate from the group of so-called carbopols, for example carbopol grades 980, 981, 1382, 2984, 5984, in each case individually or in combination. Further advantageous thickeners according to the invention are those with the INCI designation acrylates/C10-30 alkyl acrylate crosspolymer (e.g., Pemulen TR-1, Pemulen TR 2, Carbopol 1328 from NOVEON) and Aristoflex AVC (INCI: ammonium acryloyldimethyltaurate/VP copolymer).

Advantageously according to the invention the preparation according to the invention contains film formers. Film formers for the purposes of the present invention are substances of different composition which are characterized by the following property: if a film former is dissolved in water or other suitable solvents and if the solution is then applied to the skin, after the solvent has evaporated it forms a film that is used essentially to fix the light filters on the skin and thus increase the water-resistance of the product.

It is particularly advantageous to select the film formers from the group of polymers on the basis of polyvinylpyrrolidone (PVP)

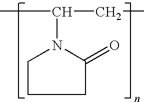

Particular preference is given to copolymers of polyvinylpyrrolidone, for example, the PVP hexadecene copolymer and the PVP eicosene copolymer, which are available under the trade names Antaron V216 and Antaron V220 from GAF Chemicals Cooperation.

Also advantageous are further polymer film formers, such as, e.g., sodium polystyrene sulfonate, which is available from the National Starch and Chemical Corp. under the trade name Flexan 130, and/or polyisobutene, available from Rewo under the trade name Rewopal PIB1000. Other suitable polymers are, e.g., polyacrylamide (Seppigel 305), polyvinyl alcohols, PVP, PVP/VA copolymers, polyglycols, acrylate/octylacralymide copolymer (Dermacryl 79). Also advantageous is the use of hydrogenated castor oil dimer dilinoleate (CAS 646054-62-8 INCI Hydrogenated Castor Oil Dimer Dilinoleate), which is available from Kokyu Alcohol Kogyo under the name Risocast DA-H, or also PPG-3 benzylethermyristate (CAS 403517-45-3), which is available under the trade name Crodamol STS from Croda Chemicals.

The oil phase of the preparation according to the invention is advantageously chosen from the group of polar oils, for example, from the group of lecithins and of fatty acid triglycerides, namely the triglycerol esters of saturated and/or unsaturated, branched and/or unbranched alkanecarboxylic acids of a chain length of from 8 to 24, in particular 12 to 18, C atoms. The fatty acid triglycerides can, for example, advantageously be chosen from the group of synthetic, semi-synthetic and natural oils, such as, for example, cocoglyceride, olive oil, sunflower oil, jojoba oil, soya oil, peanut oil, rapeseed oil, almond oil, palm oil, coconut oil, castor oil, wheatgerm oil, grapeseed oil, thistle oil, evening primrose oil, macadamia nut oil, and the like.

Also advantageous according to the invention are, for example, natural waxes of animal and vegetable origin, such as, for example, beeswax and other insect waxes, and berry wax, shea butter and/or lanolin (wool wax).

Further advantageous polar oil components can also be chosen for the purposes of the present invention from the group of esters of saturated and/or unsaturated, branched and/or unbranched alkanecarboxylic acids of a chain length of from 3 to 30 C atoms and saturated and/or unsaturated, branched and/or unbranched alcohols of a chain length of from 3 to 30 C atoms, and from the group of esters of aromatic carboxylic acids and saturated and/or unsaturated, branched and/or unbranched alcohols of a chain length of from 3 to 30 carbon atoms. Such ester oils can then advantageously be chosen from the group phenethylbenzoate, 2-phenylethylbenzoate, isopropyl lauroyl sarcosinate, phenyl trimethicone, cyclomethicone, dibutyladipate, octyl palmitate, octyl cocoate, octyl isostearate, octyl dodecyl myristate, octyldodecanol, cetearyl isononanoate, isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, stearyl heptanoate, oleyl oleate, oleyl erucate, erucyl oleate, erucyl erucate, tridecyl stearate, tridecyl trimellitate, and synthetic, semisynthetic and natural mixtures of such esters, such as, for example, jojoba oil.

In addition, the oil phase can advantageously be chosen from the group of dialkyl ethers and dialkyl carbonates, for example, dicaprylyl ether (Cetiol OE) and/or dicaprylyl carbonate, for example, that available under the trade name Cetiol CC from Cognis, being advantageous.

It is also preferred the oil component or components be from the group isoeicosane, neopentyl glycol diheptanoate, propylene glycol dicaprylate/dicaprate, caprylic/capric/diglyceryl succinate, butylene glycol dicaprylate/dicaprate, C₁₂₋₁₃ alkyl lactate, di-C₁₂₋₁₃ alkyl tartrate, triisostearin, dipentaerythrityl hexacaprylate/hexacaprate, propylene glycol monoisostearate, tricaprylin, dimethyl isosorbide. It is particularly advantageous if the oil phase of the formulations according to the invention has a content of C₁₂₋₁₅-alkyl benzoate or consists entirely thereof.

Advantageous oil components are also, for example, butyloctyl salicylate (for example that available under the trade name Hallbrite BHB from CP Hall), tridecylsalicylate (which is available under the trade name Cosmacol ESI from Sasol), C12-C15 alkylsalicylate (available under the trade name Dermol NS from Alzo), hexadecyl benzoate and butyloctyl benzoate and mixtures thereof (Hallstar AB) and/or diethylhexyl naphthalate (Hallbrite TQ or Corapan TQ from Symrise).

Any desired mixtures of such oil and wax components can also be used advantageously for the purposes of the present invention.

In addition, the oil phase can likewise advantageously also comprise nonpolar oils, for example, those which are chosen from the group of branched and unbranched hydrocarbons and hydrocarbon waxes, in particular mineral oil, Vaseline (petrolatum), paraffin oil, squalane and squalene, polyolefins, hydrogenated polysiobutenes, C13-16 isoparaffin and isohexadecane. Among the polyolefins, polydecenes are the preferred substances.

The preparations according to the invention can also advantageously comprise one or more substances from the following group of siloxane elastomers, for example, in order to increase the water resistance and/or the light protection factor of the products:
(a) Siloxane elastomers which contain the units R₂SiO and RSiO_{1.5} and/or R₃SiO_{0.5} and/or SiO₂,
   where the individual radicals R, in each case independently of one another, are hydrogen, C₁₋₂₄-alkyl (such as, for example, methyl, ethyl, propyl) or aryl (such as, for example, phenyl or tolyl), alkenyl (such as, for example, vinyl), and the weight ratio of the units R₂SiO to RSiO_{1.5} is chosen from the range from 1:1 to 30:1;
(b) Siloxane elastomers which are insoluble and swellable in silicone oil and which are obtainable by the addition reaction of an organopolysiloxane (1) which contains silicon-bonded hydrogen with an organopolysiloxane (2) which contains unsaturated aliphatic groups,
   where the quantitative amounts used are chosen such that the amount of hydrogen of the organopolysiloxane (1) or of the unsaturated aliphatic groups of the organopolysiloxane (2)
      is in the range from 1 to 20 mol % when the organopolysiloxane is non-cyclic and
      is in the range from 1 to 50 mol % when the organopolysiloxane is cyclic.

For the purposes of the present invention, the siloxane elastomer or elastomers are advantageously present in the form of spherical powders or in the form of gels.

Siloxane elastomers present in the form of spherical powders which are advantageous according to the invention are those with the INCI designation Dimethicone/Vinyl Dimethicone Crosspolymer, for example, that available from DOW CORNING under the trade names DOW CORNING 9506 Powder.

It is particularly preferred when the siloxane elastomer is used in combination with oils from hydrocarbons of animal and/or vegetable origin, synthetic oils, synthetic esters, synthetic ethers or mixtures thereof.

Particularly advantageous preparations are also obtained when antioxidants are used as additives or active ingredients. According to the invention, the preparations advantageously comprise one or more antioxidants. Favorable, but nevertheless optional, antioxidants which may be used are all antioxidants customary or suitable for cosmetic applications.

For the purposes of the present invention, water-soluble antioxidants may be used particularly advantageously, such as, for example, vitamins, e.g. ascorbic acid and derivatives thereof.

Preferred antioxidants are also vitamin E and derivatives thereof, and vitamin A and derivatives thereof.

The amount of antioxidants (one or more compounds) in the preparations is preferably from 0.001 to 30% by weight, particularly preferably from 0.05 to 20% by weight, in particular from 0.1 to 10% by weight, based on the total weight of the preparation.

If vitamin E and derivatives thereof are the antioxidant or the antioxidants, it is advantageous to choose their respective concentrations from the range from 0.001 to 10% by weight, based on the total weight of the formulation.

If vitamin A or vitamin A derivatives, or carotenes or derivatives thereof are the antioxidant or the antioxidants, it is advantageous to choose their respective concentrations from the range from 0.001 to 10% by weight, based on the total weight of the formulation.

It is particularly advantageous when the cosmetic preparations according to the present invention comprise cosmetic or dermatological active ingredients, preferred active ingredients being antioxidants which can protect the skin against oxidative stress.

Formulations according to the invention which comprise, for example, known anti-wrinkle active ingredients, such as flavone glycosides (in particular α-glycosylrutin), coenzyme Q10, retinol and ester, vitamin E and/or derivatives and the like are particularly advantageously suitable for the protection from esthetically unattractive changes in the skin, such as arise, for example, during the skin aging (such as, for example, dryness, roughness and formation of dryness wrinkles, itching, reduced refatting (e.g., after washing), visible vascular dilations (telangiectases, cuperosis), flaccidity and formation of wrinkles and fine lines, local hyperpigmentation, hypopigmentation and defective pigmentation (e.g., age spots), increased susceptibility to mechanical stress (e.g. cracking) and the like) and fatigued skin. In addition, they are advantageously suitable to counter the appearance of dry or rough skin.

The cosmetic preparations according to the invention can comprise cosmetic auxiliaries as are customarily used in such preparations, e.g., preservatives, preservative aids, complexing agents, bactericides, perfumes, substances for preventing or increasing foaming, dyes, pigments that have a coloring effect, thickeners, moisturizing or humectant substances, fillers that improve the skin feel, fats, oils, waxes or other customary constituents of a cosmetic or dermatological formulation, such as alcohols, polyols, polymers, foam stabilizers, electrolytes, organic solvents or silicon derivatives.

Preparations for the care of the skin are advantageous for the purposes of the present invention: they can be used as cosmetic sunscreen, furthermore as a make-up product in decorative cosmetics.

According to their structure, cosmetic compositions for the purpose of the present invention can be used, e.g., as protective skin cream, day or night cream, etc. It is optionally possible and advantageous to use the compositions according to the invention as the basis for pharmaceutical formulations.

It is also advantageous for the purposes of the present invention to produce cosmetic preparations, the main purpose of which is not to protect against sunlight, but which nevertheless contain a content of UV protection substances. UVA or UVB filter substances are thus usually incorporated into day creams or make-up products, for example. UV protective substances, like antioxidants and, if desired, preservatives, also represent an effective protection of the preparations themselves against spoilage.

Cosmetic preparations that are present in the form of a sunscreen agent are also favorable.

The use of the preparation according to the invention for protection against the aging of the skin (in particular against skin aging caused by UV) and as a sunscreen agent is particularly in accordance with the invention.

The use of the preparations according to the invention for preventing or reducing fine lines and wrinkles is in accordance with the invention.

The use of the preparations according to the invention for the repair of damaged skin (in particular skin stressed from washing) is in accordance with the invention.

It is advantageous according to the invention for the preparation according to the invention to have a pH value of from 5 to 8. This can be adjusted by means of the conventional acids, bases and buffer systems.

For application, the cosmetic preparations according to the invention are applied to the skin and/or the hair in sufficient quantities in the customary manner for cosmetics.

### EXAMPLES

The following examples are intended to illustrate the present invention without restricting it. Unless stated otherwise, all the quantities, proportions and percentages given are based on the weight and the total quantity or on the total weight of the preparations.

### O/W Lotion:

| | Quantity | Quantity | Quantity |
|---|---|---|---|
| Product name | [%] | [%] | [%] |
| Glycerin | 6.00 | 8.00 | 12.00 |
| Cetyl palmitate | 10.00 | 10.00 | 10.00 |
| Paraffinum liquidum | 8.00 | 8.00 | 8.00 |
| Cetyl alcohol | 3.00 | 2.00 | |
| Stearyl alcohol | | 1.00 | 3.00 |
| Cyclomethicone | 3.00 | 3.00 | 3.00 |
| Sorbitan stearate | 2.00 | 2.00 | 2.00 |
| Aluminum starch | 1.50 | | 1.50 |
| octenylsuccinate | | | |
| Phenoxyethanol | 0.80 | 0.80 | 0.80 |
| Methylparaben | 0.30 | 0.30 | 0.40 |
| Carbomer | 0.25 | 0.25 | 0.25 |
| Propylparaben | 0.10 | 0.10 | |
| Sodium hydroxide | 0.03 | 0.03 | 0.03 |
| Urea | 6.00 | 1.50 | 3.00 |
| glucosyl glycerides | 1.00 | 1.50 | 2.00 |
| Urea | 2.00 | 5.00 | 3.00 |
| Aqua | ad 100.00 | ad 100.00 | ad 100.00 |

### O/W Cream 1:

| Product name | Quantity [%] | Quantity [%] | Quantity [%] |
|---|---|---|---|
| Glycerin | 8.00 | 10.00 | 12.00 |
| Hydrogenated cocoglycerides | 5.00 | 5.00 | 5.00 |
| Stearyl alcohol | 3.50 | 3.50 | 3.50 |
| Stearic acid | 3.00 | 3.00 | 3.00 |
| Paraffinum liquidum | 3.00 | 3.00 | 3.00 |
| Cetyl alcohol | 1.50 | 1.50 | |
| Dimethicone | | 1.00 | 1.00 |
| Phenoxyethanol | 0.80 | 0.80 | 0.80 |
| Glyceryl stearate | 0.50 | 0.50 | 0.50 |
| Sorbitan stearate | 0.50 | 0.50 | 0.50 |
| PEG-100 Stearate | 0.50 | 0.50 | 0.50 |
| Methylparaben | 0.40 | 0.40 | 0.40 |
| Carbomer | 0.20 | 0.20 | 0.20 |
| Urea | 2.00 | 1.50 | 2.00 |
| Glucosyl glyceride | 1.00 | 1.50 | 2.00 |
| Urea | 2.00 | 5.00 | 3.00 |
| Hyaluronic acid | 0.10 | 0.50 | 0.30 |
| Propylparaben | 0.15 | 0.15 | 0.15 |
| Perfume | 0.03 | 0.03 | 0.03 |
| Aqua | ad 100.00 | ad 100.00 | ad 100.00 |

### O/W Cream 2:

| | Quantity | Quantity | Quantity |
|---|---|---|---|
| Product name | [%] | [%] | [%] |
| Glycerin | 6.00 | 8.00 | 10.00 |
| Cetyl alcohol | 4.00 | 4.00 | 4.00 |
| Caprylic/capric triglyceride | 3.10 | 3.10 | 3.10 |
| Paraffinum liquidum | 3.00 | 5.00 | 6.00 |
| Alcohol denat. | 3.00 | 3.00 | 3.00 |
| Cyclomethicone | | 2.90 | 2.90 |
| Dimethicone | 2.80 | | 2.80 |
| Glyceryl stearate | 2.70 | 2.70 | 2.70 |
| PEG-40 Stearate | 1.30 | 1.30 | 1.30 |
| Urea | 3.00 | 1.50 | 2.00 |
| Glucosyl glyceride | 1.00 | 1.50 | 2.00 |
| Hyaluronic acid | 0.10 | 0.50 | 0.30 |
| Tocopheryl acetate | 0.50 | 0.50 | 0.50 |
| Ethylparaben + methylparaben + | 0.50 | 0.50 | 0.50 |
| propylparaben + phenoxyparaben + | | | |
| butylparaben + isobutylparaben | | | |
| Methylparaben | 0.20 | 0.20 | 0.20 |
| Carbomer | 0.20 | 0.20 | 0.20 |
| Perfume | 0.15 | 0.15 | 0.15 |
| Propylparaben | 0.07 | 0.07 | 0.07 |
| Sodium hydroxide | 0.04 | 0.04 | 0.04 |
| Aqua | ad 100.00 | ad 100.00 | ad 100.00 |

### W/O Lotion:

| | Quantity | Quantity | Quantity |
|---|---|---|---|
| Product name | [%] | [%] | [%] |
| Methylparaben | 0.40 | 0.40 | 0.40 |
| Propylparaben | 0.15 | 0.15 | 0.15 |
| Magnesium sulfate | 0.60 | 0.60 | 0.60 |
| Glycerin | 6.00 | 8.00 | 10.00 |
| Paraffinum liquidum | 10.00 | 10.00 | 10.00 |
| Phenoxyethanol | 0.40 | 0.60 | 0.80 |
| Polyglyceryl-3 diisostearate | 2.00 | 2.50 | 2.00 |
| Isopropyl stearate | 11.25 | 11.25 | 11.25 |
| Polyglyceryl-2 dipolyhydroxystearate | 3.00 | 3.00 | 3.00 |
| Urea | 3.00 | 1.50 | 2.00 |
| glucosyl glyceride | 1.00 | 1.50 | 2.00 |
| Urea | 2.00 | 5.00 | 3.00 |
| Perfume | 0.15 | 0.15 | 0.15 |
| Aqua | ad 100.00 | ad 100.00 | ad 100.00 |

### 1. O/W Emulsions:

| | Quantity | Quantity | Quantity | Quantity | Quantity | Quantity | Quantity |
|---|---|---|---|---|---|---|---|
| Product name | [%] | [%] | [%] | [%] | [%] | [%] | [%] |
| Glycerin monostearate | 0.50 | 1.00 | 3.00 | | | 1.50 | |
| Glyceryl stearate | 2.00 | | | 1.00 | 2.00 | | 2.50 |
| citrate | | | | | | | |
| Stearic acid | | 3.00 | 0.75 | 2.00 | | | |
| PEG-40 Stearate | 0.50 | | | | | 2.00 | |
| PEG-100 Stearate | | | 1.50 | | | | |
| Lauryl methicone | | | | 0.75 | | 0.50 | |
| copolyol | | | | | | | |
| Cetyl phosphate | | | 0.75 | | 1.00 | | |
| Stearyl alcohol | | | 3.00 | | | 2.00 | 0.50 |
| Cetyl alcohol | 2.50 | 1.00 | | | 0.50 | | 2.00 |
| UVASorb ® K2A | 1.00 | | | 4.00 | | 5.00 | |
| Uvinul ® A Plus | 3.00 | 2.50 | 0.50 | 0.25 | 1.00 | 0.50 | 4.00 |
| Bis- | | 1.00 | | | | 1.00 | 0.50 |
| ethylhexyloxyphenol | | | | | | | |
| methoxyphenyl | | | | | | | |
| triazine | | | | | | | |
| Disodium phenyl | | | | 1.00 | | 2.00 | |
| dibenzimidazole | | | | | | | |
| tetrasulphonate | | | | | | | |
| Ethylhexyl triazone | 2.00 | | | 2.00 | | 2.00 | |
| Diethylhexyl butamido | | 2.00 | | | | | |
| triazone | | | | | | | |
| Ethylhexyl | | 3.50 | | 10.00 | | | |
| methoxycinnamate | | | | | | | |
| Octocrylene | | | | 5.00 | 9.00 | 7.50 | 2.50 |
| Methylene bis- | | | 2.00 | | 3.00 | | |
| benzotriazolyl | | | | | | | |
| tetramethylbutylphenol | | | | | | | |
| Ethylhexyl salicylate | | | 3.00 | | | | 5.00 |
| Drometrizol | | | 0.50 | | | 1.00 | |
| trisiloxane | | | | | | | |
| Titanium dioxide T | | 1.50 | | | 1.00 | 0.50 | |
| 805 | | | | | | | |
| Titanium dioxide MT- | 1.00 | | | 3.00 | 1.00 | | |
| 100Z | | | | | | | |
| C₁₂₋₁₅ Alkyl benzoate | | 2.50 | | | | 7.00 | 5.00 |
| Dicaprylylether | | | 3.50 | | 2.00 | | |
| Butylene glycol | 5.00 | | | 5.00 | 3.00 | | |
| dicaprylate/dicaprate | | | | | | | |
| Cetearyl isononanoate | | 4.00 | | | | 2.00 | 2.00 |
| Dimethicone | | 0.50 | 1.00 | | 2.00 | | |
| Cyclomethicone | 2.00 | | | 4.50 | | | 0.50 |
| Dimethicone/vinyl | | 4.00 | | | | | 0.50 |
| dimethicone | | | | | | | |
| crosspolymer | | | | | | | |
| PVP hexadecene | 0.50 | | | 0.50 | 1.00 | | 1.00 |
| copolymer | | | | | | | |
| Glycerin | 3.00 | 7.50 | | 7.50 | 5.00 | | 20.00 |
| Urea | | | | 5.00 | | 3.00 | |
| Hyaluronic acid | 0.10 | 0.30 | | | 0.50 | | 0.05 |
| Xanthan gum | 0.15 | | 0.05 | | | | 0.30 |
| Butylene glycol | 7.00 | 5.00 | | | | 7.00 | |
| Vitamin E acetate | 0.50 | | 0.25 | 0.50 | 0.75 | | 1.00 |
| Urea | 15.00 | 0.50 | 2.00 | 4.00 | 3.00 | 1.50 | 1.00 |
| Dioic acid | 0.25 | | | 0.20 | | 0.25 | |
| Fucogel ® 1000 | | | 1.50 | | | 5.00 | |
| Glucosyl glycerides | 2.00 | 1.00 | 0.50 | 0.1 | 1.00 | 1.50 | 3.00 |
| DMDM Hydantoin | | 0.60 | 0.40 | 0.20 | | | |
| Methylparaben | 0.15 | | 0.25 | | 0.50 | | |
| Phenoxyethanol | 1.00 | 0.40 | | 0.40 | 0.50 | | 0.60 |
| EDTA | | 0.20 | 0.35 | 0.50 | 0.02 | | 0.03 |
| Ethanol | | 2.00 | 1.50 | | 3.00 | 5.00 | 1.00 |
| Insekt Repellent 3535 | | | 5.00 | | | | |
| Perfume | 0.20 | 0.20 | | | | 0.30 | 0.40 |
| Water | ad 100 | ad 100 | ad 100 | ad 100 | ad 100 | ad 100 | ad 100 |

### Foam O/W Emulsions:

| | Emulsion 1 | | Emulsion 2 | |
|---|---|---|---|---|
| | % by | % by | % by | % by |
| Product name | weight | volume | weight | volume |
| Stearic acid | 5.00 | | 1.00 | |
| Cetyl alcohol | 5.50 | | | |
| Cetyl stearyl alcohol | | | 2.00 | |
| PEG-40 Stearate | 8.50 | | | |
| PEG-20 Stearate | | | 1.00 | |
| Caprylic/capric triglycerides | 4.00 | | 2.00 | |
| C₁₂₋₁₅ Alkyl benzoate | 10.00 | | 15.50 | |
| Cyclomethicone | 4.00 | | | |
| Dimethicone | | | 0.50 | |
| Octyl isostearate | | | 5.00 | |
| Myristyl myristate | | | 2.00 | |
| Ceresin | 1.50 | | | |
| Glycerin | 5.00 | | 10.00 | |
| Glucosyl glycerides | 1.00 | | 2.50 | |
| UVASorb ® K2A | 2.00 | | | |
| Uvinul ® A Plus | 2.00 | | 1.50 | |
| Terephthalidene | 0.50 | | | |
| dicamphorsulfonic acid | | | | |
| Drometrizol trisiloxane | 1.50 | | | |
| Ethylhexylmethoxycinnamate | 5.00 | | 4.00 | |
| Ethylhexyl triazone | | | 3.00 | |
| Octocrylene | 5.00 | | | |
| Titanium dioxide Uvinul | 1.00 | | | |
| T 805 | | | | |
| Urea | 10.00 | | 1.00 | |
| Hyaluronic acid | 0.05 | | 0.2 | |
| BHT | | | 0.02 | |
| Na₂H₂EDTA | 0.50 | | 0.10 | |
| Perfume, preservatives | q.s | | q.s | |
| Dyes, etc. | q.s | | q.s | |
| Potassium hydroxide | q.s | | q.s | |
| Water | ad 100.00 | | ad 100.00 | |
| | pH value adjusted | | pH value adjusted | |
| | to 6.5-7.5 | | to 5.0-6.0 | |
| Emulsion 1 | | 70 | | |
| Emulsion 2 | | | | 35 |
| Gas (nitrogen) | | 30 | | |
| Gas (air) | | | | 65 |

Combining the fat phase/sunscreen filter phase heated to 78° C. with the aqueous phase/sunscreen filter phase heated to 75° C. Homogenization by means of a toothed-wheeled dispersing machine (rotor-stator principle) at 65° C. Stirring for 45 min in the Becomix with gassing with helium at 1 bar with cooling to 30° C. Addition of the additives at 30° C. (perfume). Homogenization by means of a toothed-wheel dispersing machine (rotor-stator principle) at 23° C.

### Low-Viscosity to Sprayable W/O Emulsion (for Use as a Spray or an Aerosol):

| | Quantity | Quantity | Quantity | Quantity | Quantity |
|---|---|---|---|---|---|
| Product name | [%] | [%] | [%] | [%] | [%] |
| Cetyl dimethicone copolyol | 4.00 | | | 2.50 | 3.00 |
| Polyglyceryl-2 dipolyhydroxy | | | 3.00 | | 1.00 |
| stearate | | | | | |
| Isostearyl diglyceryl succinate | | | 0.75 | | 0.30 |
| Lauryl methicone copolyol | | | | 2.00 | |
| Polysorbate 65 | | | 2.00 | | 1.50 |
| PEG-100 Stearate | | | | 1.20 | 0.70 |
| Cetearyl sulfate | | | 0.25 | | 1.00 |
| Dimethicone | | 4.00 | | | 2.00 |
| Cyclomethicone | 12.00 | 20.00 | | 30.00 | 15.00 |
| UVASorb ® K2A | | | | 0.50 | |
| Uvinul ® A Plus | 3.50 | 2.00 | 0.50 | 4.00 | 0.25 |
| Bis-ethylhexyloxyphenol | 1.00 | | | 0.50 | |
| methoxyphenyl triazine | | | | | |
| Disodium phenyl | | 1.50 | | | 2.00 |
| dibenzimidazole tetrasulfonate | | | | | |
| Drometrizol trisiloxane | | | | 1.00 | |
| Ethylhexyl methoxycinnamate | 3.00 | 4.00 | | | 10.00 |
| Ethylhexyl salicylate | | | 5.00 | | 3.50 |
| Octocrylene | | 5.00 | | 4.00 | |
| Diethylhexyl butamido triazone | | 1.00 | | | 6.50 |
| Ethylhexyl triazone | 3.00 | | | | 4.00 |
| Titanium dioxide MT 100 Z | | 0.50 | 1.00 | 1.50 | 0.50 |
| Zinc oxide Z-cote | 2.00 | | | | 4.00 |
| Dicaprylyl carbonate | 5.00 | | 15.00 | | 4.00 |
| Dihexyl carbonate | | 10.00 | | | |
| C12-15 Alkyl benzoate | 7.00 | | 10.00 | | |
| Mineral oil | 10.00 | | | | 6.00 |
| Cocoglycerides | | 2.00 | | 5.00 | |
| Urea | 5.00 | 0.50 | 4.00 | 2.50 | 6.00 |
| PVP hexadecene copolymer | | 0.75 | | | 0.40 |
| Glycerin | 5.00 | 12.50 | | 5.00 | 15.50 |
| Sorbitol | 5.00 | | 10.00 | | |
| Glucosyl glycerides | 1.00 | 3.00 | 0.50 | 1.00 | 2.00 |
| α-Glucosylrutin | | | | | 0.15 |
| EDTA | | 0.15 | 0.03 | | 0.15 |
| Glycine soya | 0.75 | | | 1.50 | |
| Magnesium sulfate | 0.75 | 1.00 | | 0.45 | 1.00 |
| DMDM Hydantoin | | 0.05 | | | 0.10 |
| Phenoxyethanol | 1.00 | 0.75 | 0.50 | | 1.00 |
| Dye, oil-soluble | 0.02 | | | | |
| Perfume | 0.30 | 0.45 | 0.35 | | 0.15 |
| Water | ad 100 | ad 100 | ad 100 | ad 100 | ad 100 |

### W/O Emulsions (Creams and Lotions):

| | Quantity | Quantity | Quantity | Quantity | Quantity |
|---|---|---|---|---|---|
| Product name | [%] | [%] | [%] | [%] | [%] |
| Cetyl dimethicone copolyol | | | | 4.00 | |
| Polyglyceryl-2-dipolyhydroxy | 5.00 | 4.50 | | | 4.50 |
| stearate | | | | | |
| PEG-30 | | | 5.00 | 2.00 | |
| Dipolyhydroxystearate | | | | | |
| UVASorb ® K2A | | | | 2.50 | |
| Uvinul ® A Plus | 3.00 | 1.00 | 0.50 | 0.25 | 2.50 |
| Phenylbenzimidazole sulfonic | | 4.00 | | 2.00 | 0.50 |
| acid | | | | | |
| Ethylhexyl methoxycinnamate | | 8.00 | | 5.00 | 4.00 |
| Diethylhexyl butamido | 3.00 | 1.00 | | | 3.00 |
| triazone | | | | | |
| Ethylhexyl triazone | | | 3.00 | 4.00 | |
| Octocrylene | 7.00 | | 8.00 | | 2.50 |
| Drometrizol trisiloxane | | | 3.00 | | |
| Titanium dioxide Uvinul ® T | 2.00 | 1.00 | | | |
| 805 | | | | | |
| Titanium dioxide MT-100 TV | | | 3.00 | | 2.00 |
| Zinc oxide Z-cote ® HP1 | 2.50 | | 6.00 | | |
| Mineral oil | | | 10.0 | | 8.00 |
| Cocoglycerides | 4.00 | 6.50 | | | |
| C12-15 Alkyl benzoate | | | | 9.00 | |
| Dicaprylyl ether | 10.00 | | | | 7.00 |
| Butylene glycol dicaprylate/ | | | 2.00 | 8.00 | 4.00 |
| dicaprate | | | | | |
| Cyclomethicone | 2.00 | | | | 2.00 |
| PVP eicosene copolymer | 0.50 | | | 1.50 | 1.00 |
| Trisodium EDTA | 1.00 | | | 0.35 | |
| Ethylhexyloxyglycerin | | 0.30 | 1.00 | | 0.50 |
| Methylpropanediol | | | | | 7.50 |
| Glycerin | 5.00 | 7.50 | | 7.50 | 2.50 |
| Glucosyl glycerides | 0.50 | 2.50 | 1.50 | 0.50 | 1.00 |
| Butylene glycol | | 2.50 | 15.00 | | |
| Urea | 3.00 | 0.50 | 0.25 | 5.00 | 2.00 |
| Glycine soya | | 1.00 | 1.50 | | |
| MgSO₄ | 1.00 | 0.50 | | 0.50 | |
| Vitamin E | 0.50 | | 0.25 | | 1.00 |
| DMDM Hydantoin | | 0.60 | | 0.20 | |
| Methylparaben | 0.50 | | | 0.15 | |
| Phenoxyethanol | 0.50 | 0.40 | | 1.00 | 0.60 |
| Dihydroxyacetone | | | | 5.50 | |
| Ethanol | 3.00 | | 4.50 | | 1.00 |
| Perfume | 0.20 | | 0.20 | | 0.20 |
| Water | ad 100 | ad 100 | ad 100 | ad 100 | ad 100 |

### Hydrodispersions (for Use as a Lotion or Spray):

| | Quantity | Quantity | Quantity | Quantity | Quantity |
|---|---|---|---|---|---|
| Product name | [%] | [%] | [%] | [%] | [%] |
| PEG-40 Stearate | | 1.25 | | | |
| Cetyl alcohol | | | | 2.00 | |
| Sodium carbomer | | 0.20 | | 0.30 | |
| Acrylates/C10-30 alkyl acrylate | | | 0.40 | 0.10 | 0.10 |
| crosspolymer | | | | | |
| Xanthan gum | 0.50 | 0.30 | 0.15 | | 0.50 |
| Dimethicone/Vinyl Dimethicone | | | 5.00 | | 3.00 |
| Crosspolymer | | | | | |
| UVASorb ® K2A | | | | 3.50 | |
| Uvinul ® A Plus | 0.25 | 3.50 | 0.50 | 2.00 | 1.50 |
| Bis-ethylhexyloxyphenol | | | 0.25 | | |
| methoxyphenyl triazine | | | | | |
| Terephthalidene | | 0.20 | | | 0.50 |
| dicamphorsulfonic acid | | | | | |
| Disodium phenyl | 0.75 | | | | 1.00 |
| dibenzimidazole tetrasulphonate | | | | | |
| Ethylhexyl methoxycinnamate | | | | 5.00 | 8.00 |
| Methylene bis-benzotriazolyl | | 1.00 | | | |
| tetramethylbutylphenol | | | | | |
| Diethylhexyl butamido triazone | | | 2.00 | | |
| Ethylhexyl triazone | 4.00 | | | 4.00 | |
| Octocrylene | | 4.00 | 10.00 | | 2.50 |
| Titanium dioxide MT-100 Z | 0.50 | | 2.00 | 3.00 | 1.00 |
| C₁₂₋₁₅ Alkyl benzoate | 2.00 | 2.50 | | | |
| Butylene glycol | 4.00 | | | 6.00 | |
| dicaprylate/dicaprate | | | | | |
| Dicaprylyl carbonate | | 3.00 | | | |
| Cyclomethicone | | | 7.50 | | |
| Glucosyl glycerides | 2.50 | 3.00 | 0.50 | 1.00 | 1.00 |
| Lanolin | | | | 0.35 | |
| Urea | 1.00 | 0.50 | 4.00 | 1.50 | 2.00 |
| PVP hexadecene copolymer | 0.50 | | | 0.50 | 1.00 |
| Ethylhexyloxy glycerin | | 0.50 | 1.00 | | 0.50 |
| Glycerin | 10.00 | 7.50 | | 5.00 | 15.00 |
| Glycine soya | | 1.50 | 1.00 | | |
| Vitamin E acetate | 0.50 | 0.20 | 0.25 | 0.75 | 1.00 |
| α-Glucosylrutin | | 0.30 | | 0.25 | |
| Trisodium EDTA | | 0.30 | 0.10 | 0.20 | |
| Konkaben LMB ® | 0.20 | | | | 0.15 |
| Methylparaben | 0.50 | | | 0.15 | |
| Phenoxyethanol | 0.50 | | | 1.00 | 0.60 |
| Ethanol | 3.00 | 7.00 | 3.50 | | 1.00 |
| Perfume, dyes | q.s. | q.s. | q.s. | q.s. | q.s. |
| Water | ad 100 | ad 100 | ad 100 | ad 100 | ad 100 |

### Oil Gels:

| | Quantity | Quantity | Quantity | Quantity | Quantity |
|---|---|---|---|---|---|
| Product name | [%] | [%] | [%] | [%] | [%] |
| Octyldodecanol | 9.00 | 9.00 | | | |
| Caprylic/capric triglyceride | 9.00 | | 6.00 | | |
| C₁₂₋₁₅ Alkyl benzoate | | | | 5.00 | 8.00 |
| Butylene glycol dicaprylate/ | | 9.00 | | | 8.00 |
| dicaprate | | | | | |
| Dicaprylyl ether | 9.00 | | | 4.00 | |
| Dicaprylyl carbonate | | 7.00 | | | |
| Ethyl galactomannan (N- | 3.50 | | | | 4.00 |
| Hance ® AG 200) | | | | | |
| C₂₀₋₄₀ fatty acids + polyethylene | | | | 3.60 | |
| (Performacid ® 350) | | | | | |
| Glucosyl glycerides | 2.50 | 3.00 | 1.00 | 1.00 | 0.50 |
| Hydroxyoctacosanyl | 2.00 | | | | |
| hydroxystearate | | | | | |
| Disteardimonium hectorite | 1.00 | | | | 1.00 |
| Cetyl dimethicone | 0.50 | | 4.50 | | |
| Cyclomethicone | | | 15.00 | | 5.00 |
| UVASorb ® K2A | | | | | 1.00 |
| Uvinul ® A Plus | 1.00 | 3.50 | 2.75 | 2.00 | 0.50 |
| Ethylhexyl methoxycinnamate | 6.00 | | | 10.00 | 3.0 |
| Octocrylene | 3.50 | | 7.50 | 10.00 | |
| Ethylhexyl salicylate | | 3.50 | | | 4.00 |
| Ethylhexyl triazone | | | 2.00 | | |
| Diethylhexyl butamido triazone | | 0.50 | | 3.00 | 4.00 |
| Urea | 1.50 | 0.50 | 2.00 | 1.00 | 3.00 |
| Phenoxyethanol | 0.50 | | | | |
| Perfume, dyes | q.s. | q.s. | q.s. | q.s. | q.s. |
| Mineral oil | ad 100 | ad 100 | ad 100 | | |
| Rice oil | | | | ad 100 | ad 100 |

1.-7. (canceled)

8. A cosmetic preparation, wherein the preparation comprises a combination of (a) urea and (b) one or more glucosyl glycerides.

9. The preparation of claim 8, wherein the preparation comprises from 0.01% to 10% by weight of (b), based on a total weight of the preparation.

10. The preparation of claim 9, wherein the preparation comprises from 0.05% to 6% by weight of (b).

11. The preparation of claim 9, wherein the preparation comprises from 0.1% to 3% by weight of (b).

12. The preparation of claim 8, wherein the preparation comprises from 0.1% to 15% by weight of (a), based on a total weight of the preparation.

13. The preparation of claim 12, wherein the preparation comprises up to 10% by weight of (a).

14. The preparation of claim 12, wherein the preparation comprises up to 6% by weight of (a).

15. The preparation of claim 12, wherein the preparation comprises at least 1% by weight of (a).

16. The preparation of claim 8, wherein the preparation is free of (2-hydroxyethyl)urea.

17. The preparation of claim 8, wherein the preparation is present as at least one of a gel, an emulsion, and a dispersion.

18. The preparation of claim 8, wherein the preparation is present as an O/W emulsion.

19. The preparation of claim 18, wherein the preparation comprises one or more O/W emulsifiers selected from glyceryl stearate citrate, glyceryl stearate, cetearyl glucoside, stearic acid and salts thereof, polyglyceryl-3-methylglycose distearate, ceteareth-20, PEG-40 stearate, PEG-100 stearate, and sodium cetearyl sulfate.

20. The preparation of claim 8, wherein the preparation further comprises one or more further UV filter substances.

21. The preparation of claim 20, wherein the one or more UV filter substances comprise at least one of homomethyl salicylate 2-ethylhexyl-2-cyano-3,3-diphenylacrylate, terephthalidene dicamphorsulfonic acid, 2-ethylhexyl 2-hydroxybenzoate, an ester of cinnamic acid, a salt of phenylene-1,4-bis-(2-benzimidazyl)-3,3′-5,5′-tetrasulfonic acid, a salt of 2-phenylbenzimidazole-5-sulfonic acid, 3-(4-(2,2-bis-ethoxycarbonylvinyl)-phenoxy)propenyl)-methoxysiloxane/dimethylsiloxane copolymer, 2,4-bis-{[4-(2-ethylhexyloxy)-2-hydroxy]-phenyl}-6-(4-methoxyphenyl)-1,3,5-triazine, dioctylbutylamidotriazone, 2,4-bis-[5-1 (dimethylpropyl)benzoxazole-2-yl-(4-phenyl)-imino]-6-(2-ethylhexyl)-imino-1,3,5 triazine, 2,4,6-tribiphenyl-4-yl-1,3,5-triazine, merocyanine, titanium dioxide, and zinc oxide.

22. The preparation of claim 20, wherein the preparation comprises from 0.01% to 40% by weight of the one or more UV filter substances, based on a total weight of the preparation.

23. The preparation of claim 21, wherein the preparation comprises from 1% to 20% by weight of the one or more UV filter substances.

24. The preparation of claim 8, wherein the preparation further comprises one or more substances selected from folic acid, D-biotin, coenzyme Q10, alpha-glucosylrutin, carnitine, natural and/or synthetic isoflavonoids, genistein, flavonoids, carotenoids, creatine, creatinine, taurine, ascorbic acid and derivatives thereof, oxygen, tocopherol and esters thereof, dihydroxyacetone; long-chain and short-chain hyaluronic acid, 8-hexadecene-1,16 dicarboxylic acid, and licochalcone A.

25. The preparation of claim 8, wherein the preparation is free of p-methylbenzylidene camphor.

26. A cosmetic preparation, wherein the preparation comprises a combination of, based on a total weight of the preparation, from 1% to 15% by weight of (a) urea and from 0.1% to 6% by weight of (b) one or more glucosyl glycerides.

27. The preparation of claim 25, wherein the preparation comprises up to 10% by weight of (a).
